(19) United States
(12) Patent Application Publication
(10) Pub. No.: US 2025/0352275 A1
(43) Pub. Date: Nov. 20, 2025

(54) MEDICAL DEVICE NAVIGATION TRACKING

(71) Applicant: Ocean Orthopedics, Inc., Westport, MA (US)

(72) Inventors: Samuel Grossman, Westport, MA (US); Thomas Gamache, Westport, MA (US); James Paiva, Westport, MA (US); Raymond Parfett, Westport, MA (US)

(21) Appl. No.: 19/209,310

(22) Filed: May 15, 2025

Related U.S. Application Data
(60) Provisional application No. 63/647,715, filed on May 15, 2024.

(51) Int. Cl. A61B 34/20; A61B 17/17; A61M 25/01

(52) U.S. Cl. CPC A61B 34/20; A61B 17/1707; A61B 2034/2048; A61B 2034/2051; A61M 2025/0166

(57) ABSTRACT

A surgical system has a device and an inertial measurement unit supported by the device body. Accordingly, the inertial measurement unit is configured to produce an inertial signal as a function of the movement of the device body. The system further has a controlling unit configured to determine the location of at least a portion of the movable device body as a function of the inertial signal. The controlling unit (e.g., in the sterile field) also uses a detected stationary artificial magnetic field at a prescribed location to determine information relating to the prescribed location. Importantly, the controlling unit also is configured to automatically zero-out the inertial measurement unit during use as a function of the information relating to the prescribed location. The controlling unit also has an output to transmit a position signal having positional information relating to the movable device body.

### PRIORITY

This patent application claims priority from provisional U.S. patent application No. 63/647,715, filed May 15, 2024, entitled, “MEDICAL DEVICE NAVIGATION TRACKING,” and naming Samuel Grossman, Thomas Gamache, James Paiva, Raymond Parfett, Kenneth Prada, and Michael Gorhan as inventors, the disclosure of which is incorporated herein, in its entirety, by reference.

### FIELD

Illustrative embodiments of the invention generally relate to medical devices and, more particularly, various embodiments of the invention relate to navigating medical devices in three-dimensional space relative to patient anatomy during a medical procedure.

### BACKGROUND

Navigating surgical or medical tools, such as catheters, needles, guidewires, retraction devices, portals, probes and cutting tools, during medical procedures is an important task that requires utmost precision and accuracy. Surgeons and other health care professionals rely on various techniques and technologies to ensure the safe and effective placement of these instruments within the body. Additionally, navigation technologies are used to track and/or control the working ends of surgical robots. Undesirably, many known techniques have limitations and drawbacks.

For example, one widely used method involves fluoroscopic guidance, which employs real-time X-ray imaging to visualize the tool's position and trajectory relative to patient anatomy. While this technique provides valuable information about the device/tool's location, it exposes both the patient and the surgical team to ionizing radiation, which can be harmful with prolonged exposure. Fluoroscopic imaging is also generally limited to two-dimensional views in a chosen anatomic plane. Additionally, fluoroscopic imaging has limited soft tissue contrast, making it challenging to differentiate between various anatomical structures.

Another tool in surgical navigation involves ultrasound imaging. This non-invasive technique utilizes high-frequency sound waves to create real-time images of the body's internal structures. However, ultrasound imaging can be widely operator-dependent, and the quality of the images may vary based on the user's experience and the patient's body habitus and is often difficult to interpret. Furthermore, ultrasound can have limited penetration depth and can be obscured by hard or bony tissue, making it less suitable for navigation in deep-seated structures or obese patients.

In recent years, advanced imaging modalities such as computed tomography (CT) and magnetic resonance imaging (MRI) have been increasingly used for surgical navigation. These technologies provide detailed, three-dimensional images of the patient's anatomy, which can be integrated with specialized navigation systems. However, the use of CT scans involves exposure to ionizing radiation, and MRI can be contraindicated in patients with certain implanted devices or claustrophobia. Additionally, the integration of preoperative imaging data with real-time tracking of surgical tools can be complex and time-consuming, potentially prolonging the duration of the procedure. Further, this type of navigation can be disrupted or corrupted by metal in the field, such as metal tables or prior medical implants.

These and others also often require costly and/or bulky capital equipment, which takes precious floor space and often requires regular service and maintenance.

### SUMMARY OF VARIOUS EMBODIMENTS

In accordance with one embodiment of the invention, a surgical system has a device having a movable device body and an inertial measurement unit supported by the device body. The inertial measurement unit is configured to produce an inertial signal as a function of the movement of the device body. As such, the inertial measurement unit has an inertial sensor as well as a magnetometer. The system further has a controlling unit configured to determine the location of at least a portion of the movable device body as a function of the inertial signal. The controlling unit (e.g., in the sterile field) also uses a detected stationary artificial magnetic field at a prescribed location to determine information relating to the prescribed location. Importantly, the controlling unit also is configured to automatically zero-out the inertial measurement unit during use as a function of the information relating to the prescribed location. The controlling unit also has an output to transmit a position signal having positional information relating to the movable device body.

The system also may have an artificial magnetic source configured to produce the stationary artificial magnetic field positioned relative to the prescribed location. Among other ways, the artificial magnetic field may be produced by an electromagnet or a permanent magnet. As such, the magnetometer is configured to detect the stationary artificial magnetic field. Moreover, the magnetometer may be configured to produce a magnetometer signal with the information relating to the prescribed location. For example, the magnetometer may be configured to provide one or both of magnitude and directional information relating to the artificial magnetic field.

The controlling unit may be configured to produce the position signal using dead reckoning and sensor fusion techniques using both the magnetometer signal and inertial signal. To improve use during surgery, the controlling unit preferably is configured to automatically zero-out the inertial measurement unit on a periodic basis during use. Additionally, among other things, the body may comprise a catheter, guidewire, pointer, syringe, needle, portal, retraction system, trocar, or camera. Alternatively, the body may include a bone shaping tool or cutting tool.

In accordance with another embodiment, a surgical method inserts at least a distal portion of a surgical device into a body orifice of the patient. The surgical device has an inertial measurement unit configured to produce an inertial signal as a function of the movement of the device body. The inertial measurement unit may include a magnetometer and an inertial sensor (e.g., an accelerometer and/or gyroscope). The method produces a stationary magnetic field at a fixed and prescribed location relative to the patient in the sterile field. Next, the magnetometer detects the stationary magnetic field and determines the location of at least a portion of the surgical device as a function of the inertial signal and the stationary magnetic field. The method automatically zeros out (e.g., calibrates due to sensor drift) the inertial measurement unit during use as a function of the information relating to the prescribed location, and then forwards a position signal having positional information relating to the surgical device.

In accordance with other embodiments of the invention, a surgical system has a device with a movable device body, and first and second inertial measurement units supported by the device body. The first and second inertial measurement units respectively are configured to produce first and second inertial signals as a function of the movement of the device body. In addition, the first and second inertial measurement units respectively are prone to first and second drift. Moreover, the first inertial measurement unit is in a known position of the device body relative to the second inertial measurement unit. The 5 system also has a controlling unit configured to subtract the first inertial signal from the second inertial signal to detect at least some of the first drift and second drift and to determine the location of at least a portion of the device body. An output of the controlling unit is configured to transmit a position signal having positional information relating to the movable device body.

### BRIEF DESCRIPTION OF THE DRAWINGS

Those skilled in the art should more fully appreciate advantages of various embodiments of the invention from the following “Description of Illustrative Embodiments,” discussed with reference to the drawings summarized immediately below.

FIG. 1 schematically shows a medical device navigation system to assist navigating a medical device in a medical procedure in accordance with illustrative embodiments.

FIGS. 2A and 2B schematically and respectively show medical devices having one and more than one inertial measuring unit in accordance with various embodiments.

FIG. 3 schematically shows a controlling unit in accordance with illustrative embodiments.

FIGS. 4A-4G schematically show various embodiments of FIG. 1. Specifically,

FIG. 4A schematically shows pointer device implementations of FIG. 1 in accordance with illustrative embodiments.

FIG. 4B schematically shows an exemplary use of a system configured in accordance with illustrative embodiments in the sacrum joint.

FIG. 4C schematically shows a pointer device implementation of FIG. 1 used for glenoid pins and shaping devices in accordance with illustrative embodiments.

FIG. 4D schematically shows a pointer device implementation of FIG. 1 for spinal pedicle screw targeting, probing, drilling, tapping, and screw insertion in accordance with illustrative embodiments.

FIG. 4E schematically shows a pointer device implementation of FIG. 1 for bone tunnel targeting, probing, drilling, tapping, and anchor insertion in accordance with illustrative embodiments.

FIG. 4F schematically shows a flexible/steerable device implementation of FIG. 1 in accordance with illustrative embodiments.

FIG. 4G schematically shows a cutting device implementation of FIG. 1 in accordance with illustrative embodiments.

FIG. 4H schematically shows a sub-millimeter and post-surgical review implementation of FIG. 1 in accordance with illustrative embodiments.

FIG. 5 schematically shows details of an inertial measuring unit in accordance with illustrative embodiments.

FIG. 6 schematically shows an initiation and storage container configured in accordance with illustrative embodiments.

FIG. 7 shows a process of using the navigation system in accordance with illustrative embodiments.

FIG. 8 schematically shows an example of a patient having an artificial magnetic field generation device in accordance with illustrative embodiments.

### DESCRIPTION OF ILLUSTRATIVE EMBODIMENTS

In illustrative embodiments, a medical instrument can be more carefully navigated into position within a patient. To that end, the instrument has an inertial measuring unit (IMU) that communicates with a controlling unit to determine the location of that IMU. Preferably, the controlling unit is at the point of care and/or within the sterile field. The IMU is configured to mitigate the effects of sensor drift within the IMU, improving navigation results. Details of illustrative embodiments are discussed below.

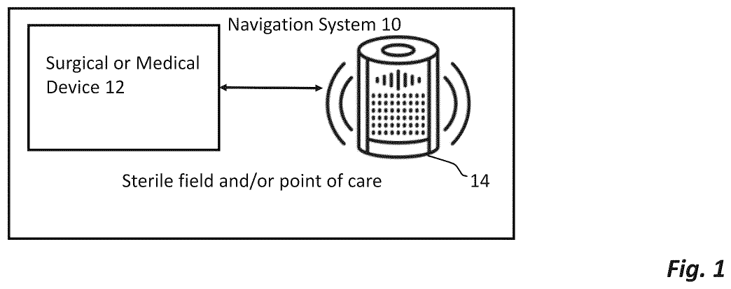

FIG. 1 schematically shows a medical device navigation system 10 to assist navigating a medical device 12 in a medical procedure in accordance with illustrative embodiments. As shown, the system 10 has a medical device 12 (also referred to as an “instrument” or “surgical instrument”) with a portion that is to be inserted into a patient (not shown in FIG. 1). For example, the device 12 may include a pointer with a distal tip that is inserted into an opening formed in the patient. To manage navigation, the device 12 has a device body fixedly supporting one or more inertial measuring units (“IMU 16”) that communicate with a controlling unit 14 (e.g., a “puck”). Among other ways, after an initialization process (discussed below), the IMU 16 forwards movement signals to the controlling unit 14, which tracks the IMU 16 and, consequently, aids in real-time or near real-time navigation inside or near the patient.

As known by those in the art, the IMU 16 is an electronic device that measures and reports angular velocity, acceleration, and the position and orientation of the device 12 using one or more of accelerometers, gyroscopes, and one or more other sensors. In preferred embodiments, that other sensor includes one or more magnetometers. Among other things, the IMU 16 may be implemented as a wireless inertial measurement sensor (WIMU). The controlling unit 14 may provide IMU power remotely, wirelessly, wired, or other to the IMU 16 and the IMU 16 provides IMU data (e.g., remotely, wirelessly, wired, or similar) to the controlling unit 14. Alternatively, the IMU may be powered some other way, such as with an internal battery.

The IMU 16 beneficially may track instrument positions for navigating the device 12 through the patient's body, as well as for malpractice analysis, teaching residents (telestration), and data collection/data mining for improved outcomes and continuous learning applications (e.g., for artificial intelligence or machine learning training). This system 10 may also be used in conjunction with other digital medical tools or measurement devices to triangulate and or calculate other real-time data. For example, the IMU 16 may help spatial computing and motion tracking of tools and implants to better quantify outcomes and analyze provider fatigue.

In one embodiment, a flex circuit provides the electrical connections between the IMU 16 and the controlling unit components. In another embodiment, the electrical connections may be provided by conventional wiring surrounded by a sterilizable jacket. The electrical connections may be separated from the controlling unit 14 by a connector affixed to an outside wall of the controlling unit 14. This can allow a device 12 with flexible connections to be modularly separated from the controlling unit 14 and disposed while the controlling unit 14 is sanitized and repackaged, following a medical or surgical procedure. In one embodiment, the flexible connection may include one or more light sources (e.g., laser or LED), radar sources, or user controls. Indeed, other embodiments may wirelessly connect the IMU 16 with the controlling unit 14.

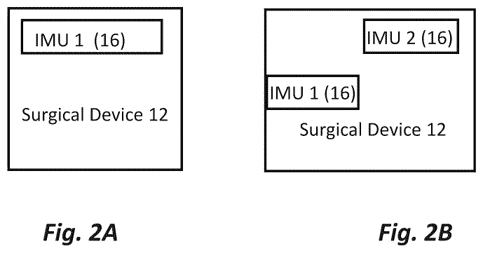

FIG. 2A schematically illustrates one embodiment of the surgical device 12 configured with a single IMU 16. In this configuration, the IMU 16 is rigidly mounted to (e.g., within or outside) a movable portion of the device body and is responsible for tracking the movement and orientation of the device 12 during use. As described in further detail below, this embodiment utilizes a stationary artificial magnetic field strategically positioned at a known, fixed location within the surgical environment. The magnetometer within the IMU 16 detects this magnetic field, enabling the system 10 to periodically recalibrate or zero-out accumulated sensor drift by comparing detected magnetic characteristics to expected reference values. This process allows the system 10 to maintain localization accuracy over time, despite inherent sensor limitations such as gyroscopic drift or accelerometer bias.

FIG. 2B schematically shows another embodiment of the surgical device 12, which incorporates two or more IMUs 16. These IMUs 16 may be distributed along different portions of the device body or mounted in geometrically distinct orientations (e.g., mounted orthogonally to each other). This configuration enables redundant motion sensing and provides cross-referencing capabilities between sensors. By comparing the output of the multiple IMUs 16, the system 10 can detect inconsistencies or anomalies indicative of sensor drift. This enables the controlling unit 14 to perform real-time correction and calibration without relying on external references, such as a magnetic field. Techniques such as sensor fusion, internal geometric modeling, or dynamic motion constraint analysis may be employed to enhance accuracy and robustness, particularly in complex or magnetically noisy environments.

These alternative configurations-single versus multiple IMUs 16-offer different trade-offs in terms of system complexity, calibration methodology, and resilience to environmental disturbances. The selected embodiment may depend on surgical use case, device size constraints, and required precision.

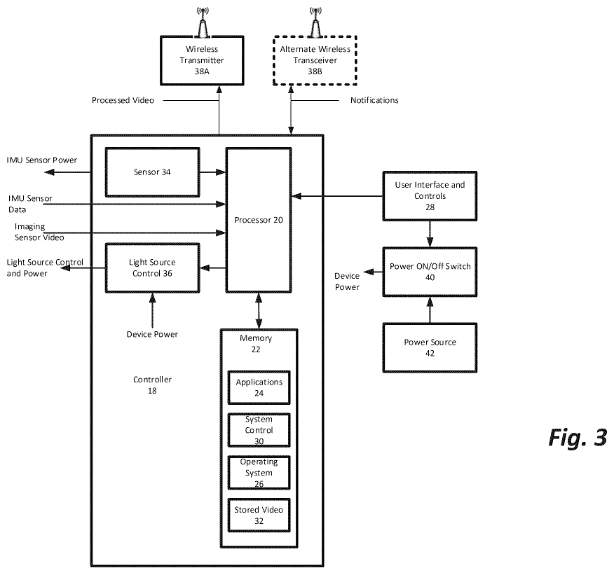

FIG. 3 schematically illustrates further detail of the controlling unit 14 in accordance with various illustrative embodiments of the surgical system 10. The controlling unit 14 is implemented as an integrated electronic module housing multiple computational, sensing, and communication subsystems, each configured to perform dedicated functions during operation.

At its core, the controlling unit 14 includes a controller 18 comprising one or more processors 20-such as microprocessors, digital signal processors (DSPs), or application-specific integrated circuits (ASICs)—along with associated memory resources (referred to as “memory 22”). Among other things, the memory 22 may include both volatile memory, such as random-access memory (RAM), and non-volatile storage, such as flash memory, solid-state drives (SSDs), or embedded storage modules. These computing and memory resources 22 are configured to support a wide array of system functions for operation of the surgical navigation system 10.

In particular, among other things, the memory resources enable the real-time execution of signal processing algorithms, including the fusion of sensor data from accelerometers, gyroscopes, and magnetometers; the implementation of drift correction routines; and the execution of calibration and zeroing procedures. Additionally, the memory 22 supports data buffering, intermediate computation, and real-time control tasks, which may involve complex processing pipelines that must operate with low latency and high reliability in the surgical environment.

Beyond processing tasks, the memory 22 also stores the operating system 26 and firmware responsible for controlling the behavior of the processor 20 and coordinating peripheral subsystems. It may include application-level software 24, such as instrument tracking modules, user interface logic, communication protocols, and safety monitors. In some embodiments, the memory 22 may also contain stored video 32, including video streams received from external sources (e.g., endoscopes or cameras) or video rendered from sensor data for real-time display or post-operative review. The system 10 may optionally archive procedural video logs or instrument motion data for quality control, teaching, or medico-legal documentation.

Moreover, the memory 22 may be used to manage system control parameters 30, configuration files, reference field signatures (such as magnetic field models), and device-specific profiles. These optional elements allow the system 10 to adapt to different surgical workflows, instruments, or patient anatomies. In this way, the memory 22 acts not only as a computational workspace, but also as a long-term repository for operating configurations, updates, logs, and analytics, enabling the controlling unit 14 to serve as a centralized and intelligent hub for surgical device navigation and coordination.

In addition to its computational components, the controller 18 also supports various auxiliary sensors and peripherals (both referred to with reference number “34”). These may include, for example, a microphone for capturing audio data-useful for voice-command interfaces or surgical annotations—and a light source 36 for illumination or signaling purposes. The light source 36 may be configured to provide visible or infrared (IR) light, depending on operational needs such as endoscopic visibility or system status indication.

The controlling unit 14 further incorporates a wireless communication interface, including a radio frequency (RF) transmitter and associated antenna 38A. This interface is designed to transmit processed video signals, referred to as wireless transmitted video 404, to one or more remote display or visualization systems. Examples include surgical video monitors, augmented reality (AR) headsets, and virtual reality (VR) devices, which may be used by the operating surgeon or support staff for enhanced spatial awareness and guidance during a surgical procedure. The system 10 also may have an alternative wireless transceiver 38B that also communicates with the processor 20 (e.g., for notifications).

In one embodiment, the transmitting antenna is fully enclosed within the housing of the controller 18 or controlling unit 14 itself, enabling a compact and sterilizable form factor without external protrusions. The wireless transmission system is engineered to maintain high-fidelity, low-latency video streaming over distances exceeding 10 meters, ensuring robust connectivity even in large or complex operating rooms. This transmission may utilize standards such as Wi-Fi (e.g., IEEE 802.11ac or ax), ultra-wideband (UWB), or specialized medical-grade wireless protocols optimized for minimal interference and secure data handling.

Taken together, these features enable the controlling unit 14 to serve as a central hub for sensor integration, data processing of IMU signals, and real-time surgical visualization, while preserving mobility and minimizing tethered connections in the sterile field. In preferred embodiments, the controlling unit 14 is similar to and incorporates various relevant features like that shown in co-pending U.S. patent application Ser. No. 18/896,724, filed Sep. 25, 2024, with the title, “Method of Augmenting Tissue,” and assigned to Ocean Orthopedics, Inc., of Westport, MA, the disclosure of which is incorporated herein, in its entirety, by reference.

User interface and controls 28 may include various manual interfaces designed to allow surgical personnel to control and adjust parameters associated with the navigation of the surgical device 12 using the IMU 16. These controls 28 enable full operation of the controlling unit 14, which processes IMU data to determine the spatial position and orientation of the device 12 during a procedure. The configuration controls are designed to be usable within the sterile field, and are optimized for gloved operation, with careful consideration given to tactile responsiveness and error prevention.

One primary control may include a power ON/OFF switch or button 40, which activates or deactivates the navigation subsystem. To prevent accidental shutdowns during surgery, the system 10 may incorporate design elements, such as a raised protective barrier or “fence” around the button 40, or a hinged mechanical cover that must be deliberately lifted before the control can be accessed. In some embodiments, the power control may function only as the power-on switch 40, with no power-off functionality once the system 10 is active. In such configurations, shutting down the device 12 may require a separate action-such as receiving a shutdown signal from an external user device, like a tablet or workstation, or physically removing the power source 42 from the unit.

The user configuration controls may also include dedicated controls for managing the behavior of the IMU 16 during navigation. For example, a manual calibration input may allow the user to initiate a re-zeroing procedure during the operation, aligning the IMU 16 with a known reference, such as a stationary artificial magnetic field. Another control may launch automatic calibration/re-zeroing during use at some periodic or non-periodic interval. Additional controls may be provided to adjust the sensitivity of the IMU response, including parameters that affect motion filtering, noise rejection, and the interpolation of position data from raw inertial signals. Other controls may enable the operator to switch between different navigation modes-such as free-hand tracking, constrained movement assistance, or robotic path-following-depending on the clinical workflow and the nature of the surgical task.

To supplement the physical controls, the system 10 may also include a software interface accessible via a computer, tablet, or dedicated app. This interface provides a more detailed set of configuration options, allowing clinical staff to monitor IMU performance, update firmware, adjust advanced parameters, and visualize the real-time movement of the surgical device 12 in a graphical format. The software interface may also be used for logging, diagnostic routines, and remote control in settings where manual interaction with the device 12 is impractical.

Together, the physical controls and software interface offer a comprehensive and flexible means for surgical personnel to manage the navigation system in real time, ensuring that the surgical device 12 maintains high positional accuracy and responsiveness, with minimal effects from sensor drift, throughout the procedure.

As noted, the controlling unit 14 also is configured to track navigation using signals received from the IMU(s) 16. Among other ways, the controlling unit 14 may use dead reckoning after initializing the position of the IMU 16. Specifically, in some embodiments, the IMU 16 provides the required data that the controlling unit 14 utilizes for dead reckoning by measuring linear acceleration and angular velocity. These measurements enable the controller 18 to track the instrument's movement in three-dimensional space. From a known starting point, the controlling unit 14 computes changes in position and orientation over time by integrating the acceleration to estimate velocity and then integrating the velocity to estimate displacement. This method, however, is susceptible to cumulative errors known as drift, primarily due to the noise inherent in IMU sensors and the integration process of velocity and position.

Some embodiments may display tracking information as an overlay on an underlying imaging device, such as an ultrasound device.

In illustrative embodiments, the device 12 also uses a time-of-flight sensor to facilitate navigation. Specifically, as known by those in the art, time-of-flight sensors capture detailed spatial information about the surroundings, enabling the controlling unit 14 to detect fixed features in the patient and track their movement relative to the device 12. This capability allows for a method known as “visual odometry,” where the position and orientation of the device 12 are refined by observing the changes in these features over time. By mapping these changes against the IMU's data, the system can correct discrepancies and/or fill in gaps in the IMU-derived trajectory, thereby enhancing accuracy and adjusting for drift.

To further optimize navigation, the controlling unit 14 may employ advanced algorithms, such as the Kalman filter or complementary filter, for sensor fusion. These algorithms integrate the diverse data streams from the IMUs 16 and time-of-flight sensors, adjusting estimates (continuously or not continuously) to minimize errors. The result is a more robust navigation system 10 capable of precise positioning and better adaptability to dynamic environments. This constructive collaboration between diverse types of sensors not only improves the reliability of dead reckoning but also enhances the overall operational efficiency of the navigation system 10.

Navigating the device 12 through or onto a human or animal body using a combination of an IMU 16, and time-of-flight sensors involves a multi-step algorithm designed to integrate data from these sensors to achieve accurate and reliable navigation. The process may start by the system initializing the system 10 by calibrating the IMU 16 and time-of-flight sensors. Calibration ensures that the sensors are synchronized in time and aligned spatially. The instrument's initial position and orientation are established based on the available data.

As the device 12 moves, the IMU 16 continuously records accelerations and angular velocities to calculate changes in position and orientation. Simultaneously, the time-of-flight sensors capture images or laser scans of the environment. This data includes distances to various objects and features detectable within the sensor's range. The algorithm processes the data from the time-of-flight sensors to detect distinctive features in the environment (like edges, corners, or specific features). These features are matched against a database of known features (if available) or tracked over time to determine their movement relative to the device 12.

Using the IMU data, the system 10 applies dead reckoning to estimate the instrument's new position based on velocity and direction changes. This estimate is then refined using the feature data from the time-of-flight sensors. The algorithm calculates the relative movement of detected features to correct the drift that occurs in the IMU data. As noted above, techniques such as Kalman filtering, or particle filtering can be employed to fuse the data from both sensors and update the instrument's estimated state (position, velocity, orientation).

As the device 12 navigates, the algorithm continuously adjusts the estimates based on new sensor data. It minimizes error by weighing the confidence in data from each sensor type, adjusting dynamically to changes in sensor performance or patient internal conditions. This might involve increasing reliance on time-of-flight data when IMU data is uncertain due to sensor noise or other disturbances. With updated and refined position information, the system can make navigational decisions. The time-of-flight sensors provide critical 10 data about obstacles, enabling the device 12 to navigate more efficiently. In some cases, the system 10 might perform loop closure checks to identify if the device 12 has returned to a previously visited location. This helps in correcting cumulative navigation errors and updating the environmental map used for navigation.

In other embodiments, the controlling unit 14 and its controller 18 may utilize an artificial magnetic field source positioned at a known, fixed location on or within the patient's body to facilitate enhanced calibration of the IMU 16. This artificial magnetic field may be generated by a passive magnet, or an active field-emitting element embedded in or affixed to the patient's anatomy in a consistent, reproducible manner. By incorporating a magnetometer as part of the IMU 16, the system 10 can detect this artificial magnetic field and use it as a reference signal to correct for cumulative drift in the accelerometer and gyroscope data over time.

This calibration process leverages the known position and signature of the artificial magnetic field to re-zero or realign the IMU readings, ensuring that minor deviations in angular velocity and linear acceleration-common sources of error in inertial tracking-do not accumulate and degrade positional accuracy during the procedure. The correction may be performed periodically, such as at predetermined intervals or surgical milestones, or not periodically, in response to detected anomalies or environmental disturbances. In some embodiments, sensor fusion algorithms are employed to integrate data from the magnetometer, gyroscope, and accelerometer, optimizing the accuracy in positional data and dynamically correcting for drift without requiring explicit operator intervention.

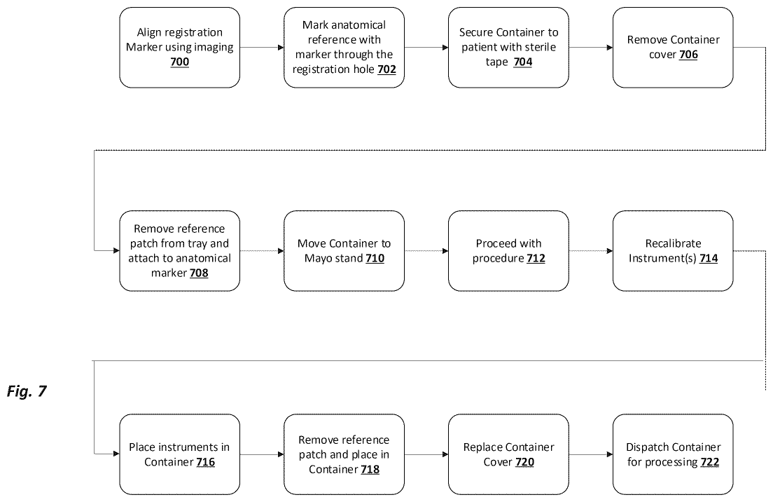

The use of a magnetic reference point located on or within the patient enables highly localized, in situ calibration-minimizing the need for external tracking hardware and preserving the flexibility and sterility of the surgical field. Additional technical details of this calibration technique, including example algorithms and magnetic field modeling strategies, are provided below with reference to FIG. 7.

As noted above, the computing device preferably is in the sterile field and/or at the point of care. As known by those in the art, the sterile field typically is an area for operating on a patient. In this context, an operating room may be divided into a non-sterile field and a sterile field. As known by those in the art, in an operating room, the division between sterile and nonsterile fields is essential for maintaining asepsis during surgical procedures. This division helps prevent the transmission of bacteria and other pathogens that could lead to infections.

To that end, the sterile field includes the area where the surgery takes place. It often is defined by the surgical drapes and/or another/other barriers that are placed around the patient on the operating table. These drapes create a barrier that separates the surgical site from the rest of the operating room. All the instruments, equipment, and supplies used within this area are sterilized. Personnel who enter the sterile field are required to wear sterilized gowns, gloves, caps, and masks to maintain the sterility. Only those directly involved in the surgical procedure, such as surgeons, scrub nurses, and surgical technicians, typically enter the sterile field.

Sterile or semi-sterile field can also include specific areas inside treatment rooms in a clinic or office setting. Often these areas are used in these sites of care to perform interventional treatments.

The nonsterile field includes areas of the operating room that are outside the immediate surgical site. This includes the anesthesia station, equipment bays, and, in the prior art, computer stations used to monitor the patient's vitals and other important parameters. Personnel in this area, such as anesthesiologists and circulating nurses, generally do not enter the sterile field and typically do not wear sterile gowns. They handle non-sterile tasks such as coordinating with other departments, managing patient records, and supplying non-sterilized equipment as needed.

The operating room may have transitional areas, which can be designated between the sterile and nonsterile fields. Personnel use these areas to change from nonsterile to sterile attire or vice versa. Protocols for handwashing, gowning, and gloving are rigorously followed in these areas to maintain the integrity of the sterile environment. The strict separation of these areas and adherence to protocols ensures that the sterile field is free from any contamination that could potentially lead to infections, safeguarding patient health during surgical procedures.

The operating room is one well-known area known as the “point of care.” “Point of care” more generally refers to the location or environment where medical care is directly administered to a patient. This concept emphasizes the delivery of health care services at the time and place of patient need, which can significantly enhance the timeliness, precision, and effectiveness of medical treatments and interventions. In addition to an operating room, the point of care also includes clinics, ambulatory surgical centers, physicians' offices, mobile treatment vehicles or temporary facilities, on a battlefield or other non-hospital site (among other places).

In a doctor's office, point of care might refer to the use of diagnostic tools and testing kits that provide instant results, enabling doctors to make quicker diagnoses and treatment decisions during a consultation. For instance, tests like glucose monitoring for diabetics, rapid strep tests, or INR testing for patients on anticoagulants are commonly used. This setting allows for a quick transition from diagnosis to the initiation of treatment, enhancing patient convenience and care continuity. On battlefields, accident sites (e.g., an automobile accident) or in non-hospital environments, point of care takes on a critical role due to the urgent need for medical interventions in potentially life-threatening situations. Portable medical kits, emergency treatment units, and mobile diagnostic tools are utilized to administer immediate care. Medical personnel in these settings are often equipped to perform rapid assessments, emergency procedures, and initial stabilization to save lives or prevent further injuries until the patient can be transported to a hospital.

Point of care also extends to community health settings, home care, and remote areas where traditional hospital resources are unavailable. Technologies such as telemedicine, mobile health apps, and portable diagnostic devices enable healthcare providers to deliver care directly to patients in their community or home, improving access to healthcare services for underserved populations.

In each of these scenarios, point of care is designed to minimize delays in treatment, reduce the need for multiple visits or referrals, and improve overall health outcomes by integrating care delivery into the patient's immediate environment. This approach is becoming increasingly important in healthcare as technologies advance and the emphasis on patient-centered care grows.

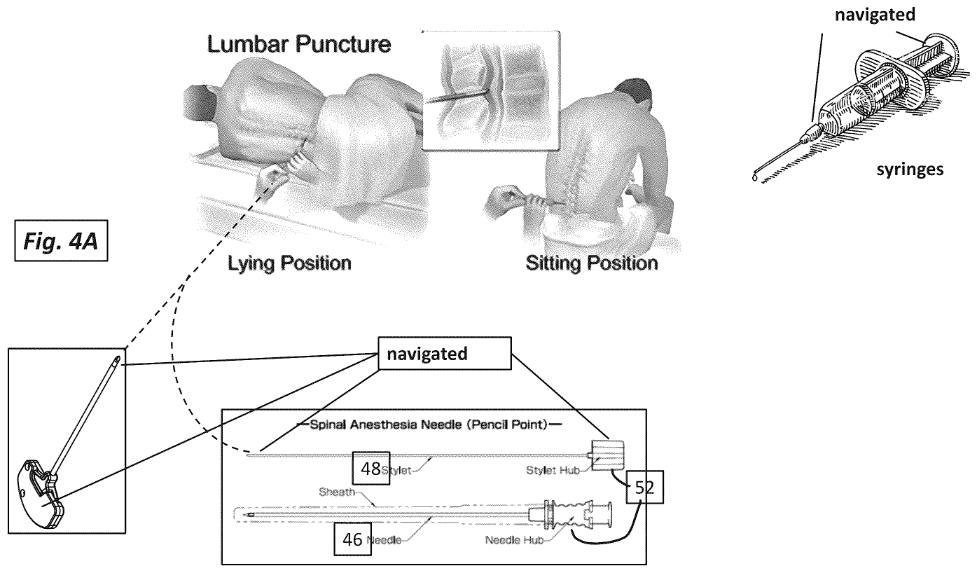

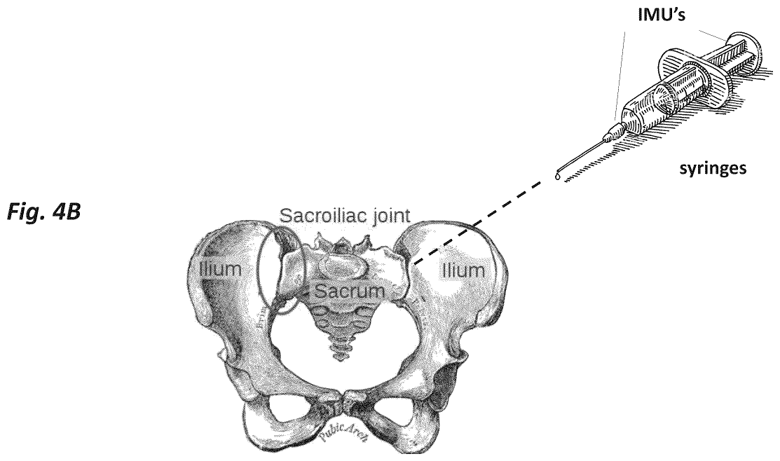

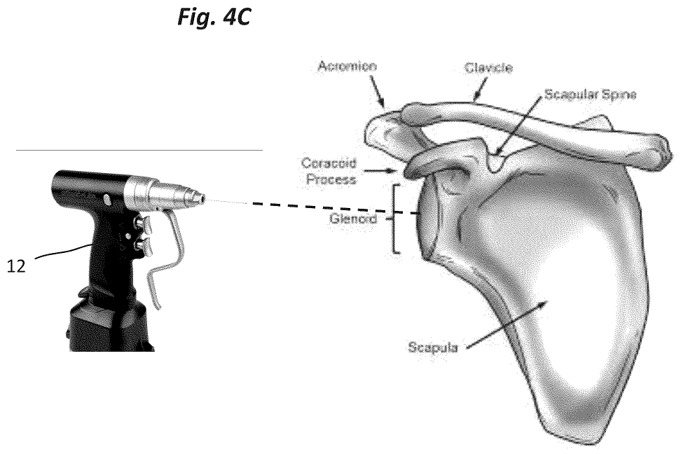

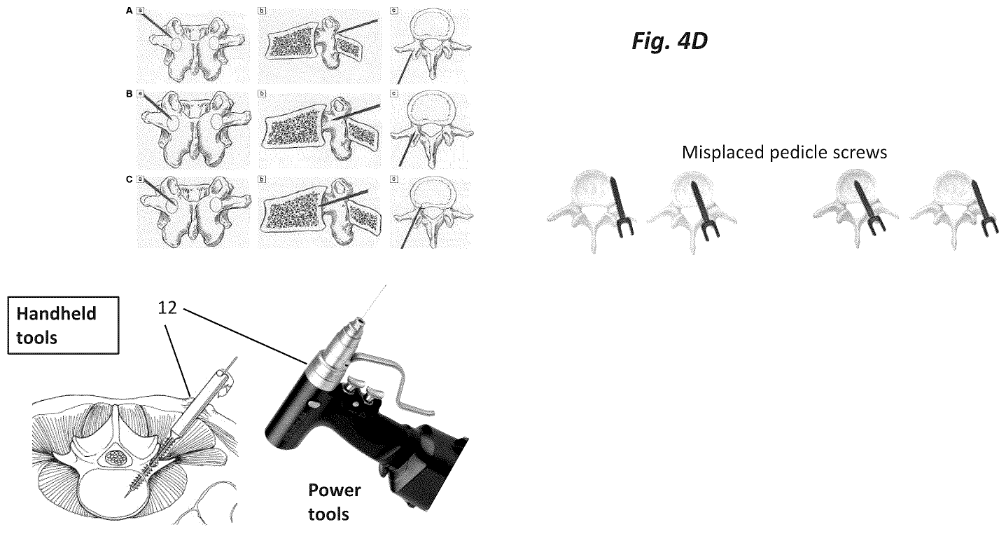

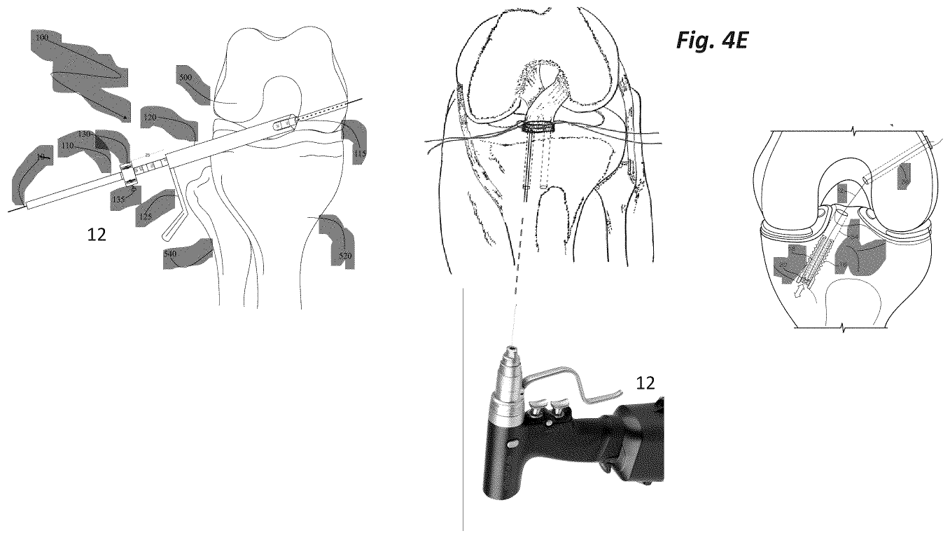

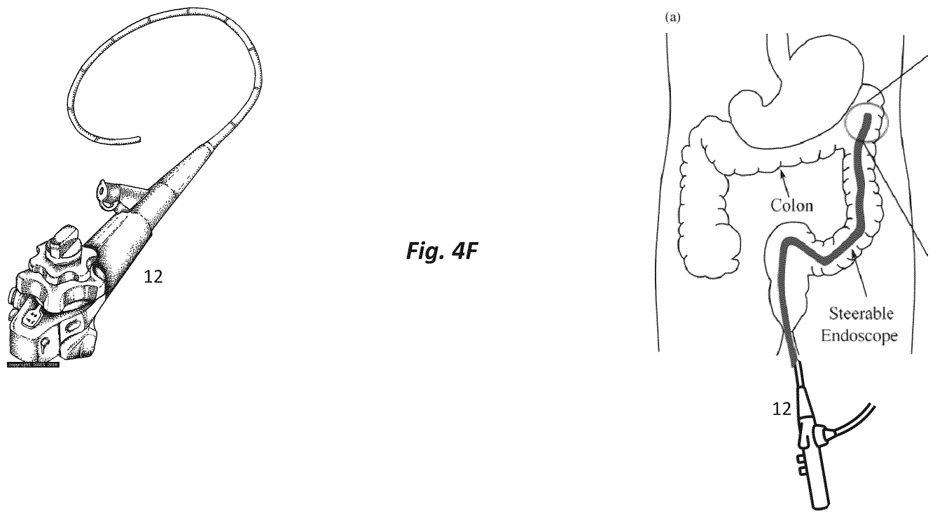

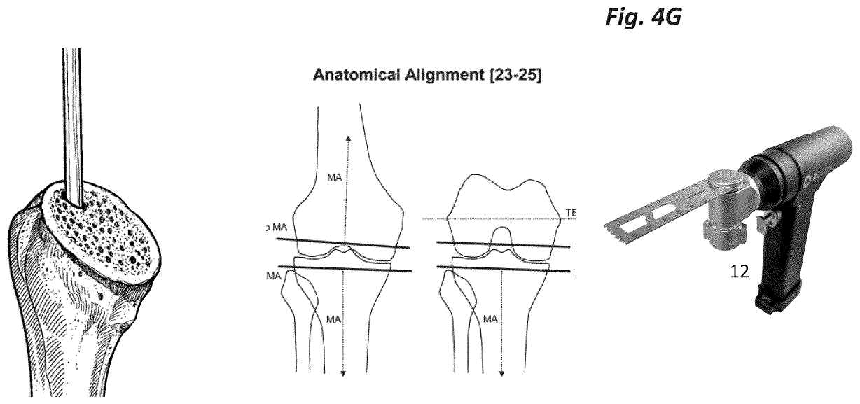

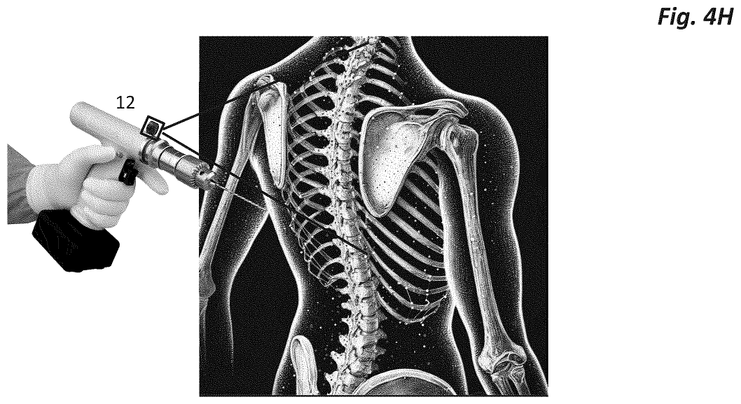

FIGS. 4A through 4H schematically illustrate a variety of exemplary use cases for the surgical navigation system 10 described herein, in accordance with illustrative embodiments. These figures demonstrate the system's versatility and its ability to integrate with different surgical devices 12 and workflows across a range of clinical applications.

FIG. 4A shows a pointer device 12 implementation of the system 10, as initially introduced in FIG. 1. In the context of medical and surgical procedures, a pointer device 12 typically refers to an instrument used to guide, direct, or locate anatomical targets with precision. Often, it defines a spatial path—for example, a straight line between two anatomical landmarks or along the axis of a surgical tool. Pointer devices 12 may be passive, such as alignment instruments, or active, such as sensor-equipped guides that report positional data in real time.

One common medical example of a pointer device 12 is a sheath 44 or cannula. These tubular devices 12 are used to facilitate the safe and accurate insertion of secondary tools such as catheters, guidewires, needles 46, or optical probes. The sheath 44 acts as both a protective conduit and a spatial guide, reducing trauma to surrounding tissue while maintaining precise access to target regions. In procedures such as catheterization or epidural anesthesia, a sheath 44 may serve to preserve vascular or spinal access, allowing multiple instruments to be exchanged efficiently through a single insertion point.

In its pointer role, the sheath 44 may work in concert with instruments such as spinal anesthesia needles 46, stylets 48, and guidewires. For instance, FIG. 4A shows an example of a spinal anesthesia needle 46 with a stylet 48 and stylet hub 52, illustrating the coordinated function of such tools in navigating toward a precise anatomical location. The sheath's ability to stabilize and guide the movement of these internal components is important, especially in procedures where direct line-of-sight navigation is not feasible.

Although the sheath 44 is a representative example, it is not limiting. Other pointer devices 12 compatible with the system 10 include trocars, syringes, and even flexible or rigid probes. In each case, the system 10 may be adapted to support device-specific geometries and procedural goals.

The IMU 16 may be embedded at various positions along the pointer device 12, including at the distal end, the proximal end, or an intermediate location depending on the application. In some embodiments, the pointer device 12 itself may also integrate all or part of the system's computing functionality, eliminating the need for a separate external controlling unit 14.

FIG. 4B illustrates the use of the system 10 in an orthopedic application involving the sacroiliac (SI) joint, a common target in pain management and joint stabilization procedures. The IMU-enhanced pointer device 12 enables precise targeting and trajectory control in this anatomically complex region.

FIG. 4C shows the pointer device 12 configured for use with glenoid pins and shaping devices. In this embodiment, IMUs 16 may be embedded at multiple locations on the device 12, including the handle or shaft, allowing for real-time tracking of angular orientation and tool trajectory. The pointer device 12 in this configuration features a central lumen for guidewire delivery, ensuring that the guidewire travels in a controlled path along the device's longitudinal axis, enhancing accuracy and consistency during pin placement or shaping operations.

FIGS. 4D and 4E further demonstrate use of the system 10 in spinal and orthopedic workflows. FIG. 4D shows the pointer device 12 adapted for procedures involving pedicle screw targeting, drilling, tapping, and screw insertion-tasks requiring millimeter-level accuracy to avoid neural and vascular structures. These may include, for example, handheld tools or power tools. FIG. 4E presents a related application involving bone tunnel targeting and anchor placement, as is common in reconstructive or ligament repair surgeries. In some or all of these embodiments, the system 10 may optionally incorporate time-of-flight sensors to enhance localization and steerability/trackability. These sensors provide complementary data to the sensors in the IMU 16, enabling depth estimation or distance measurements relative to anatomical features or surgical landmarks.

FIG. 4F illustrates a flexible or steerable endoscope implementation, another representative pointer device 12 that benefits from IMU integration. In this embodiment, IMUs 16 and/or time-of-flight sensors may be placed at or near the distal tip, proximal base, or along the shaft of the endoscope. This configuration enables fine-grained tracking of endoscope movement in three dimensions, improving safety and navigation within complex internal cavities. In some variations, radar-based sensors may also be incorporated to further enhance spatial awareness.

FIG. 4G shows a cutting device 12 implementation, such as a surgical drill or burr, incorporating the IMU-based navigation system 10. This embodiment supports dynamic monitoring of cutting trajectory and rotational orientation, which is especially useful when operating near critical structures.

FIG. 4H illustrates a system 10 used for sub-millimeter surgical review or post-operative assessment, with enhanced visualization via virtual anatomical rendering. This rendering may be generated from preoperative or intraoperative imaging data, such as CT scans, MRI, or intraoperative 3D ultrasound, and represented as a point cloud or volumetric model. The surgical device 12, shown here with an integrated time-of-flight sensor, is visualized in relation to the point cloud, enabling surgeons to assess alignment, depth, and completeness of a procedure in real time or during retrospective analysis.

In some embodiments, as noted and suggested above, the body of the surgical device 12 can include an instrument, inserter, or guide configured to facilitate the placement of a tertiary body-such as an implant, screw, or marker-within or on a patient. The instrument may include a tool directly manipulated by the user to engage and position the tertiary body, for example, a screwdriver, grasper, or forceps. In other embodiments, the body may incorporate an inserter specifically adapted to retain and controllably deploy the tertiary body into a desired anatomical location. Such inserters can be configured for engagement with interbody spinal cages, tissue anchors, or radiopaque markers. Alternatively, the body may include a guide element configured to provide alignment, orientation, or depth control for accurate placement. The guide may be realized as a drill guide, cutting template, or navigation-assist feature, and may interface with imaging or robotic systems. In some configurations, the device may further comprise a delivery mechanism such as a cannula or catheter adapted for minimally invasive insertion of the tertiary body, including but not limited to stents, brachytherapy seeds, or laparoscopically deployed components.

In summary, FIGS. 4A-4H, among other things, collectively highlight the flexibility and scalability of the system 10 across a wide spectrum of surgical tools and clinical settings. Whether implemented in rigid guides, flexible instruments, or powered devices, the IMU-based tracking system-optionally augmented with ToF, radar, or magnetic field references-offers real-time positional awareness and precise guidance in even the most complex surgical environments.

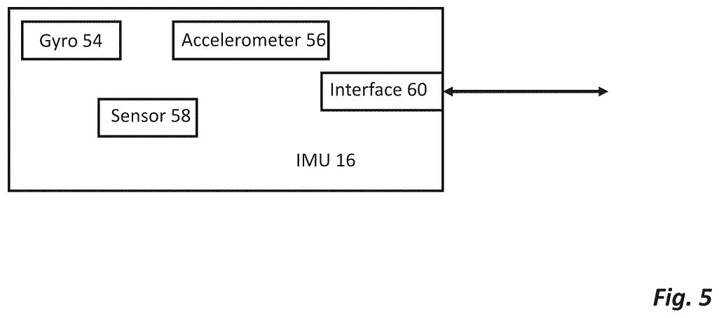

FIG. 5 schematically illustrates a block diagram of an IMU 16 suitable for use in the surgical navigation system 10 described in prior figures. The IMU 16 is a compact multi-sensor module that integrates three core sensing components: at least two inertial sensors-a gyroscope 54, an accelerometer 56—as well as a magnetometer 58. These components operate in coordination to provide continuous, real-time data on the orientation, acceleration, and movement of the surgical device 12 to which the IMU 16 is affixed.

The gyroscope 54 measures angular velocity across one or more axes, allowing the system 10 to track how the surgical device 12 rotates in space. The accelerometer 56 detects linear acceleration, which can be used to infer translational motion as well as gravitational orientation. The magnetometer 58, meanwhile, detects ambient magnetic fields-including Earth's natural magnetic field or controlled artificial magnetic fields introduced into the surgical environment. This enables the system 10 to determine heading and to correct for drift that may accumulate in the gyroscope 54 and accelerometer 56 over time. Specifically, the magnetometer 58 may detect the direction, the amplitude, or both characteristics of the artificial magnetic field. When combined in a sensor fusion framework, these three sensing modalities work in concert to generate a robust motion profile, enabling high-precision localization even in dynamic, confined, or magnetically complex surgical settings.

In the embodiment shown, the IMU 16 also includes a dedicated communication interface 60 that enables it to transmit sensor data to the controlling unit 14. This interface 60 may use a wired connection (e.g., I²C, SPI, or UART) or a wireless link (e.g., Bluetooth Low Energy or UWB), depending on the implementation and whether the IMU 16 is embedded in a reusable, disposable, or sterilizable component. The communication interface 60 may support not only raw sensor output but also pre-processed data, such as filtered position estimates, diagnostic flags, or time-stamped sensor events.

The IMU 16 may have other components, which are not shown. It should be noted that their omission is for simplicity purposes and not to suggest that some embodiments may not have other IMU components.

The integration of this IMU 16 into the surgical device 12 allows the controlling unit 14 to interpret the physical motion of the device 12 in real time. Through periodic or continuous recalibration-such as by referencing a fixed artificial magnetic field placed on or near the patient—the IMU 16 can maintain accurate orientation and position data throughout a procedure with minimal intervention by the surgeon. This also enables features like virtual navigation overlays, augmented reality guidance, and precise robotic assistance, all of which depend on accurate device 12 localization for safe and effective surgical outcomes.

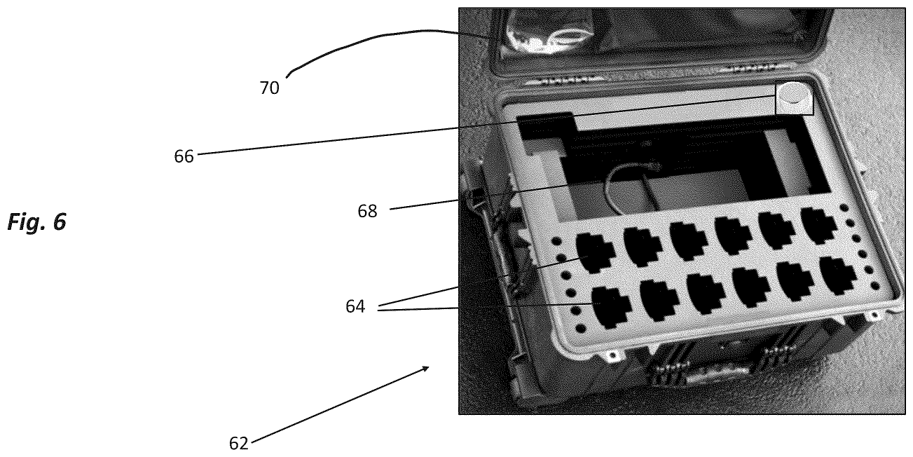

Illustrative embodiments may be distributed to hospitals or surgical centers as a kit having one or more instruments within a single structure. To that end, FIG. 6 schematically illustrates a sophisticated initiation and storage container 62 designed to manage the storage, transportation, and initialization of IMUs 16 embedded in medical devices 12. This storage container 62 helps ensure that the devices 12 are ready for immediate use upon deployment and as such, can be a handy form factor for point of care sites outside of a controlled setting (e.g., outside of a hospital setting). The storage unit is engineered with a body having multiple slots 64, each spaced at a precise distance to accommodate multiple devices 12 securely. This arrangement not only protects the devices 12 during transport but also facilitates rapid deployment by keeping them organized and easily accessible. The precise distance aids in managing the distance between IMUs 16. Upon opening a cover 70 on the container 62, the IMUs 16 within the devices 12 automatically begin the boot-up process, allowing for real-time tracking and data collection from the moment of activation. Alternatively, the boot up process may be initiated manually via a switch, button, or other interface to the container 62.

The initiation process is further enhanced by the integration of a strong magnet 66 within the container 62, which serves as a local point of reference. Note that this magnet 66 may be used alone and/or in addition to the noted magnet 66 on or in the patient for calibration during use. This magnet 66 is important for maintaining a point of reference for the sensors. It also can help in calibrating the instruments' IMUs 16 in relation to their environment, ensuring that the sensor data is accurately aligned and translated in real-time during procedures. This feature is particularly valuable in dynamic medical settings where precision and speed are critical, and it helps in reducing setup times and improving the reliability of the instruments' outputs.

Power management is another important aspect addressed by the design of the container 62. the battery 66 within the unit keeps the IMUs 16 in a continuously charged state, ready for immediate use. The battery 66 can charge the IMUs 16 through both wired and wireless methods, offering flexibility in how the devices 12 are maintained. This dual charging capability ensures that the devices 12 are always operational, regardless of the availability of external power sources, making the system 10 highly reliable and efficient for medical professionals in various settings. Some embodiments may use kinetic charging technology.

FIG. 7 shows a process of deploying a surgical navigation system 10 that utilizes the noted container 62 of FIG. 6, which holds IMU-equipped surgical devices 12. It should be noted that some of the steps may be performed in a different order than that shown, or at the same time. Moreover, one or more steps may be omitted in certain contexts. Those skilled in the art therefore can modify the process as appropriate.

The process begins with the alignment of a radiopaque registration marker built into the container's controlled-geometry packaging (step 700). This marker may be visualized using imaging techniques such as fluoroscopy, CT, or X-ray, allowing the clinician to align the container 62 precisely with the desired anatomical region. The built-in geometry ensures that the internal orientation of the marker is known and consistent, serving as the basis for registration.

After the container 62 is correctly aligned, the clinician marks the patient's anatomy through a registration hole in the container 62 (step 702). This mark defines a precise point on the patient's body that corresponds to the internal reference seen on imaging, effectively anchoring the system's coordinate space to the patient's anatomy. The container 62 is then secured to the patient using sterile tape or a comparable surgical-grade fixation method to ensure positional stability during the remainder of the setup process (step 704).

The next step involves removing the cover of the container 62 (step 706). This action powers on and initializes the IMUs 16 embedded in or attached to the surgical devices 12 inside the container 62. Upon activation, the IMUs 16 undergo a series of initial calibration routines, including gyroscopic and accelerometric baseline alignment, as well as magnetometer referencing using either ambient or the artificial magnetic fields as described earlier. This automated initialization prepares each device 12 for active tracking.

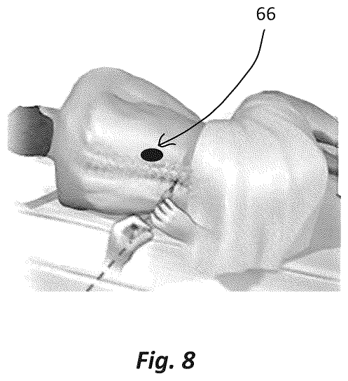

The process then may remove a reference patch from the container's internal tray and affixed it to the anatomical site marked earlier (step 708). This patch, which may include embedded tracking features and/or magnetic elements (e.g., an electromagnet 66 or permanent magnet 66), becomes the central spatial reference point for navigation during the procedure. FIG. 8 schematically shows a simplified view of the magnet 66 on a patient's back as an example. After affixed, the container 62 may be moved to a sterile field platform, such as a Mayo stand, making the devices 12 easily accessible for surgical use (step 710). At this point, the surgical devices/devices 12 may be actively tracked and spatially registered relative to the anchored reference patch.

With the devices 12 and reference system initialized, the clinician may proceed with the surgical procedure (step 712), relying on continuous real-time data from the IMUs 16 embedded within or attached to the surgical devices 12. These IMUs 16 provide a stream of data corresponding to the instruments' spatial orientation, acceleration, and angular velocity. The data is transmitted to a controlling unit 14, which processes the signals to compute a dynamic estimate of the instrument's position and heading in the patient's anatomy. This tracking information forms the basis for live feedback to the surgical team, often through graphical overlays or augmented visualizations.

As noted above, to ensure long-term accuracy during the procedure, the system 10 includes mechanisms for recalibrating the IMU 16 to correct for sensor drift and accumulated error (step 714). Specifically, in some embodiments, the system 10 recalibrates the IMU 16 as a function of the information relating to a prescribed anatomical location having the prior noted artificial magnetic field. For example, in one embodiment, recalibration is performed by touching the device 12 to a reference patch previously affixed to a known anatomical location (i.e., a prescribed anatomical location). This physical contact allows the system 10 to reset the instrument's position and orientation to a known reference frame. However, in other embodiments, automatic recalibration may be performed without requiring physical contact; in other words, with no interaction by the surgeon. In these implementations, the system 10 relies on data from the magnetometer 58 integrated within the IMU 16.

Specifically, the magnetometer 58 detects local magnetic fields, such as the noted artificial magnetic field introduced into the surgical environment. In this system 10, the stationary artificial magnetic field is generated at a fixed location on or within the patient's body. This field may be produced by a strategically positioned permanent magnet 66 or an electromagnet 66 configured to emit a field with known characteristics. As the surgical device 12 moves through space within the patient, the magnetometer 58 continuously captures vector data-representing the direction and/or magnitude of the magnetic field at the instrument's current location. When the device 12 enters a region where the detected field matches the known signature of the artificial magnetic source, the controlling unit 14 may interpret this as a recalibration opportunity. Specifically, since the position and orientation of the field are predetermined and stable, the system 10 can use that moment to correct or reset errors that may have built up in the IMU 16 over time. That is, the device 12 can be recalibrated automatically based on the match between what is sensed and what is expected.

Using this field detection, the controlling unit 14 can re-zero the instrument's heading and orientation by comparing the measured magnetic vector to a stored reference vector associated with the artificial field. This process can occur periodically, such as at predefined time intervals or procedural stages, or not periodically, in response to real-time conditions such as threshold drift detection or proximity to the reference field. The recalibration can be executed without interrupting the surgical workflow and does not require direct interaction from the surgeon, thereby preserving sterility and procedural efficiency.

The magnetometer's readings may be further enhanced through integration with sensor fusion algorithms. Sensor fusion refers to the mathematical and algorithmic combination of data from the accelerometer 56, gyroscope 54, and magnetometer 58 to produce a unified, high confidence estimate of the instrument's position and orientation. Each sensor alone has limitations: gyroscopes 54 suffer from long-term drift, accelerometers 56 are sensitive to short-term noise, and magnetometers 58 can be distorted by nearby ferrous materials. Through fusion, however, the strengths of each sensor are leveraged while their weaknesses are minimized.

For example, gyroscopes 54 offer high-precision angular velocity readings over short durations, enabling responsive motion tracking. Accelerometers 56 can detect both dynamic motion and static gravitational vectors, making them useful for determining tilt or inclination. Magnetometers 58 provide an absolute frame of reference by indicating orientation relative to the magnetic field. In a fusion framework-often implemented via Kalman filters, complementary filters, or adaptive modeling—the system 10 uses a predictive model of motion to estimate the instrument's state and continuously corrects it using incoming sensor measurements. When magnetometer data is deemed reliable, it is given higher weight to anchor heading estimates; when it is distorted, its influence is reduced, allowing the gyroscope 54 and accelerometer 56 to temporarily dominate.

The combination of these techniques enables dead reckoning, whereby the instrument's position is inferred over time by integrating prior position, velocity, and orientation data. While dead reckoning inherently accumulates error, the periodic recalibration using the magnetometer 58-whether initiated by physical proximity to a reference field or triggered algorithmically-allows the system 10 to limit drift and maintain accuracy throughout long and complex procedures. This integrated approach ensures that the surgeon receives continuously accurate feedback, allowing real-time course corrections and precise anatomical targeting.

As the device 12 moves through the patient's anatomy, the controlling unit 14 may generate and transmit a position signal to connected video displays or augmented reality devices. To that end, the controlling unit 14 may have an output with a wired or wireless connection to one or more display devices. As such, this signal may transmit to the display device(s) for a visual display that aids the surgeon. Among other things, this may be rendered as a visual overlay on preoperative imaging such as CT or MRI, or as a standalone virtual representation. The resulting feedback allows the clinician to visualize the device 12 in anatomical context, assess tool orientation relative to critical structures, and make intraoperative adjustments with confidence.

Upon completing the procedure, the devices 12 are returned to the container 62 (step 716), and the reference patch is removed from the patient and placed back into the container 62 as well (step 718). After the components are returned, the container's cover is replaced, which powers down the IMUs 16 and protects the system 10 for post-operative handling (step 720). Finally, the sealed container 62 is dispatched for processing (step 722). Processing may include sterilization, recharging, data offloading, or preparation for reuse in future surgical cases.

This structured and closed-loop workflow enhances surgical precision, reduces setup and cleanup complexity, and ensures that IMU-based tracking is initialized, calibrated, maintained, and securely shut down in a controlled and reproducible manner. The process supports a wide range of surgical devices 12 and techniques while preserving sterility and streamlining intraoperative coordination.

Accordingly, the single-IMU embodiment (or a multi-IMU embodiment that uses this technique) advantageously provides the ability to perform automatic, intraoperative recalibration with a fixed artificial magnetic field, such as one placed on or within the patient's body (or even external to the patient's body but in or outside the surgical field). Unlike traditional navigation systems-which often require manual recalibration steps, external tracking arrays, or visual line-of-sight confirmations to restore positional accuracy—this approach allows the system 10 to autonomously correct for drift without any explicit user input or workflow interruption. As the device 12 moves into proximity with the known magnetic reference field, the system 10 detects the expected magnetic vector and magnitude using the onboard magnetometer 58 and automatically re-zeros or reorients the IMU 16 based on this known signature. This recalibration can occur periodically (e.g., at defined intervals) or not periodically, such as when subtle errors in estimated position begin to exceed a threshold, as detected by the sensor fusion algorithms.

This provides at least two benefits: it maintains navigational accuracy over time, even during long or repetitive procedures, and it does so in a way that is seamless, sterile, and transparent to the surgical team. This contrasts sharply with systems that depend on external infrastructure, require physical docking of the tool to a base station, or demand surgical pauses to reorient tracking. By embedding the calibration mechanism into the surgical field itself-using a passive, non-powered magnetic field source—this system 10 delivers robust, maintenance-free correction that enhances reliability and reduces cognitive and procedural burden for the clinician.

Rather than using the artificial magnetic source 66, other embodiments may incorporate multiple IMUs 16 on or within the body of the surgical device 12. For example, a first IMU 16 may be positioned near the distal end of the device body-close to the tool tip—while a second IMU 16 may be mounted proximally, near the handle or at a stable portion of the device 12. These units work in tandem to improve spatial awareness, reduce cumulative drift, and allow more nuanced tracking of the device's motion over time.

To address sensor drift, the multi-IMU system 10 can use the known spatial relationship between the two IMUs 16 to compare their outputs. By subtracting or otherwise analyzing the difference between their respective inertial signals, the controlling unit 14 can detect inconsistencies that are attributable to drift rather than true motion. For instance, if both IMUs 16 are rigidly fixed to the same portion of the device body and no relative motion is expected between them, a divergence in their outputs may indicate that one or both units have accumulated drift. The controlling unit 14 can use this information to correct the device's overall positional estimate, improving accuracy without requiring external references.

In certain implementations, the system 10 may use a kinematic model of the device body to support drift correction. By modeling the expected movement of the device 12-whether as a rigid body, a tool with constrained axes, or a flexing structure—the controlling unit 14 can determine whether discrepancies between the IMU outputs are physically plausible or are likely due to sensor degradation. This modeling approach allows the system 10 to apply real-time corrections in a way that preserves responsiveness and precision during use.

Other embodiments may apply one or more filtering techniques, such as Kalman filters, extended Kalman filters, or complementary filters, to blend the signals from both IMUs 16 and remove long-term drift. These filters can be configured to give more weight to the more stable or reliable IMU 16 at a given time, depending on sensor health, environmental conditions, or signal history.

Although in many cases the two IMUs 16 are mounted in fixed positions relative to one another, that need not always be the case. In some embodiments, the second IMU 16 may be mounted on a movable element of the device 12-such as an articulating arm, steerable shaft, or flexible section-allowing the system 10 to track not only the device's global position but also its internal articulation or bending profile. This is particularly valuable in procedures using flexible tools, where shape reconstruction is necessary for accurate navigation.

By incorporating multiple IMUs 16 in this way, the system 10 enhances reliability and redundancy in navigation, providing the surgeon with a stable and accurate representation of the device's position and orientation even in the presence of dynamic movement, challenging anatomical geometries, or long procedural durations.

### Example: Dual-IMU System in Catheter Navigation

In one representative use case, the dual-IMU 16 configuration is employed in a steerable catheter system 10 used for navigating tortuous vasculature, such as during intracardiac ablation or neurovascular interventions. A first IMU 16 may be positioned at the proximal handle of the catheter, where the surgeon manipulates the device 12 outside the body. A second IMU 16 is embedded near the distal tip of the catheter, either within the steerable segment or just proximal to it.

Because catheter shafts are flexible and undergo complex bending as they navigate the vasculature, the tip and handle often move differently. To account for this, system 10 uses data from both IMUs 16-along with a calibrated geometric model of the catheter shaft—to estimate the catheter's shape and orientation in real time. The system does not expect the IMUs to match, but it does expect their relative motion to follow predictable patterns based on known catheter mechanics. If the distal IMU 16 exhibits motion-especially rotational drift—that diverges over time from what the model predicts based on handle movement, the system 10 interprets this as sensor drift. The controlling unit 14 detects such divergence by comparing the actual tip data with the expected tip behavior derived from the handle IMU and the catheter model. When discrepancies exceed acceptable thresholds, the system recalibrates the tip position and updates the displayed orientation, providing continuous, corrected feedback to the clinician.

In some embodiments, this approach may allow the generation of a live virtual rendering of the catheter's path through the body, even without fluoroscopic imaging. The enhanced localization allows the surgeon to navigate safely and efficiently, reducing radiation exposure and improving targeting accuracy for therapeutic interventions.

### Example: Dual-IMU System in Spinal Screw Placement

In another example, the system 10 is configured for use in pedicle screw placement during spinal surgery, such as lumbar or thoracic fusion. Here, the surgical tool may be a pedicle probe, awl, or drill guide, and the IMUs 16 are both affixed to the rigid body of the tool. One IMU 16 is located near the distal shaft, close to the entry point into the vertebra, while the other is located at the proximal end, near the handle or interface with a robotic or mechanical arm.

Because of the high sensitivity required in pedicle screw trajectories—where even slight angular deviation can risk breaching the spinal canal-accurate inertial tracking is important. The system 10 periodically or continuously compares the readings from the proximal and distal IMUs 16. If either sensor begins to drift due to accumulated bias or motion artifacts, the controlling unit 14 detects the inconsistency using differential analysis and corrects the trajectory estimate in real time. This ensures that the surgical navigation remains stable and accurate, particularly during prolonged or forceful tool manipulation. Additionally, by applying a biomechanical model of rigid tool movement and constraints from the surrounding anatomy, the system 10 filters out spurious deviations and maintains the tool's alignment relative to preoperative imaging. The surgeon receives ongoing positional feedback through the display or heads-up visualization, allowing precise targeting of the pedicle and confident placement of the implant.

This multi-IMU embodiment yields unexpected improvements over conventional surgical navigation systems, such as those that rely heavily on external infrastructure, such as optical tracking arrays, electromagnetic field generators, or mechanical linkages. These prior systems typically require line-of-sight, rigidly fixed reference frames, or carefully maintained environmental conditions, which can be disrupted by surgical personnel, fluid accumulation, patient movement, or metallic interference.

In contrast, the use of two inertial measurement units in known relation to one another-combined with real-time signal comparison and drift modeling-allows for autonomous drift detection and correction without reliance on external hardware. The approach is notably resilient in constrained or dynamic environments, such as crowded operating rooms or procedures involving large instrumentation or robotic arms, where traditional external tracking methods may become obscured or impractical. Additionally, the dual-IMU system 10 enables continuous internal verification of sensor integrity, offering built-in redundancy and robustness that are typically achieved, if at all, only with added complexity and cost. The integration is also highly compact and compatible with sterilizable designs, which is often a major limitation in optical or field-based tracking technologies. As such, this architecture provides a cleaner, more integrated, and lower-maintenance alternative for accurate surgical navigation-one that aligns well with modern demands for mobility, reliability, and intraoperative adaptability.

Some embodiments may enable recalibration or zero-out processes without a reference to the patient anatomy. For example, the system 10 can be implemented using two cameras positioned at a fixed reference point-such as mounted on or near a trocar or surgical portal. These cameras can be arranged (e.g., orthogonally) to provide complementary views of the instrument as it passes through the reference point. As the surgical instrument moves through this field of view, the system detects its passage using visual markers placed along the instrument shaft, as well as by recognizing the shape of the instrument's tip. This dual-sensor setup allows the system to recalibrate the IMU dynamically, using both the X and Y perspectives to increase spatial accuracy. The camera feeds support several key functions:
Image recognition of depth markings on the instrument to determine insertion depth.
Shape recognition to identify the geometry of the instrument tip.
Angle estimation of the instrument relative to the portal.
XY localization of the instrument within the surgical field.

With sufficient depth markings along the instrument, the system can infer the position of the tip based on known instrument specifications, potentially eliminating the need for direct tip recognition. This approach allows for recalibration with varying levels of confidence, depending on the number and clarity of the detected markers and the instrument's known geometry.

The embodiments of the invention described above are intended to be merely exemplary; numerous variations and modifications will be apparent to those skilled in the art. Such variations and modifications are intended to be within the scope of the present invention as defined by any of the appended claims.

What is claimed is:

1. A surgical system comprising:
a device having a movable device body;
an inertial measurement unit supported by the device body, the inertial measurement unit configured to produce an inertial signal as a function of the movement of the device body, the inertial measurement unit comprising a magnetometer and an inertial sensor; and
a controlling unit configured determine the location of at least a portion of the movable device body as a function of the inertial signal, the controlling unit using a detected stationary artificial magnetic field at a prescribed location to determine information relating to the prescribed location, the controlling unit configured to automatically zero-out the inertial measurement unit during use as a function of the information relating to the prescribed location,
the controlling unit comprising an output to transmit a position signal having positional information relating to the movable device body.

2. The surgical system of claim 1 wherein the controlling unit is configured to be at the point of care or in a sterile field.

3. The surgical system of claim 1 wherein the prescribed location is on or in a patient's body.

4. The surgical system of claim 1 wherein the artificial magnetic source comprises an electromagnet.

5. The surgical system of claim 1 wherein the magnetometer is configured to produce a magnetometer signal with the information relating to the prescribed location, the controlling unit configured to produce the position signal using dead reckoning and sensor fusion techniques using both the magnetometer signal and inertial signal.

6. The surgical system of claim 1 wherein the controlling unit is configured to automatically zero-out the inertial measurement unit on a periodic basis during use.

7. The surgical system of claim 1 wherein the body comprises a catheter, guidewire, pointer, syringe, needle, portal, retraction system, trocar, or camera.

8. The surgical system of claim 1 wherein the body comprises a bone shaping tool or cutting tool.

9. The surgical system of claim 1 wherein the magnetometer is configured to provide both magnitude and directional information relating to the artificial magnetic field.

10. The surgical system of claim 1 further comprising an artificial magnetic source configured to produce the stationary artificial magnetic field positioned relative to the prescribed location, the magnetometer configured to detect the stationary artificial magnetic field.

11. The surgical system of claim 1 wherein the body comprises an instrument, inserter, or guide used in placement of a tertiary body in a patient's body.

12. A surgical method for a patient, the surgical method comprising:
inserting at least a distal portion of a surgical device into a body orifice of the patient, the surgical device having an inertial measurement unit configured to produce an inertial signal as a function of the movement of the device body, the inertial measurement unit comprising a magnetometer and an inertial sensor;
producing a stationary magnetic field at a fixed and prescribed location relative to the patient, the prescribed location being a sterile field having the patient;
detecting, by the magnetometer, the stationary magnetic field;
determining the location of at least a portion of the surgical device as a function of the inertial signal and the stationary magnetic field;
automatically zeroing out the inertial measurement unit during use as a function of the information relating to the prescribed location;
forwarding a position signal having positional information relating to the surgical device.

13. The method of claim 12 wherein producing a magnetic field comprises using an electromagnet to produce the magnetic field.

14. The method of claim 12 wherein determining the location comprises applying sensor fusion techniques, dead reckoning techniques, or both fusion and dead reckoning techniques to the inertial signal and the detected stationary magnetic field.

15. The method of claim 12 wherein automatically zeroing out comprises automatically zeroing out the inertial measurement unit on a periodic basis during use.

16. The method of claim 12 wherein detecting comprises detecting, by the magnetometer, the magnitude, directional information, or both the magnitude and directional information of the magnetic field.

17. A surgical system comprising:
a device having a movable device body and configured to produce a first inertial signal as a function of the movement of the device body;
a first inertial measurement unit supported by the device body and configured to produce a first inertial signal as a function of the movement of the device body, the first inertial measurement unit being prone to first drift;
a second inertial measurement unit supported by the unit and configured to produce a second inertial signal as a function of the movement of the device body, the second inertial measurement unit being prone to second drift, the first inertial measurement unit being in a known position relative to the second inertial measurement unit; and
a controlling unit configured to subtract the first inertial signal from the second inertial signal to detect at least some of the first drift and second drift and to determine the location of at least a portion of the device body,
the controlling unit comprising an output to transmit a position signal having positional information relating to the movable device body.

18. The surgical system of claim 17 wherein the controlling unit is configured to model the device body to correct at least some of the first and second drift.

19. The surgical system of claim 17 wherein the controlling unit is configured to apply a filter to correct at least some of the first and second drift.

20. The surgical system of claim 17 wherein the first inertial measurement unit is in a fixed position relative to the second inertial measurement unit.

21. The surgical system of claim 17 wherein the first inertial measurement unit is in a movable position relative to the second inertial measurement unit.
